(19) United States
(12) Patent Application Publication
(10) Pub. No.: US 2016/0045442 A1
(43) Pub. Date: Feb. 18, 2016

(54) STABLE PHARMACEUTICAL COMPOSITIONS OF MESALAMINE

(71) Applicant: CADILA HEALTHCARE LIMITED, Ahemdabad Gujarat (IN)

(72) Inventors: Brij Khera, Princeton, NJ (US); Sushrut Krishnaji Kulkarni, Ahmedabad (IN); Hothur R. Kiran, Ahmedabad (IN)

(21) Appl. No.: 14/826,125

(22) Filed: Aug. 13, 2015

Related U.S. Application Data
(63) Continuation-in-part of application No. 14/593,067, filed on Jan. 9, 2015.

(30) Foreign Application Priority Data
Aug. 13, 2014 (IN) 2602/MUM/2014

(51) Int. Cl. A61K 9/48; A61K 31/606

(52) U.S. Cl. CPC A61K 9/4808; A61K 31/606; A61K 9/4825

(57) ABSTRACT

The present invention relates to stable pharmaceutical compositions of mesalamine. The composition of the invention is a capsule dosage form filled with a tablet. The invention also relates to process for preparing such compositions. The invention specifically relates to a composition of mesalamine wherein the composition is devoid of any reducing sugar or sugar alcohol.

### CROSS-REFERENCE TO RELATED APPLICATIONS

This application claims priority to Indian Provisional Application No. 2602/MUM/2014 filed on Aug. 13, 2014 and Indian Patent Application No. 2602/MUM/2014, filed on Aug. 12, 2015. This application also claims priority as a continuation-in-part of U.S. patent application Ser. No. 14/593,067, filed on Jan. 9, 2015.

### FIELD OF THE INVENTION

The present invention relates to stable pharmaceutical compositions of mesalamine. The composition of the invention is a capsule dosage form filled with a tablet. The invention also relates to a process for preparing such compositions. The invention specifically relates to a composition of mesalamine wherein the composition is devoid of any reducing sugar or sugar alcohol.

### BACKGROUND OF THE INVENTION

The active compound aminosalicylic acid (in particular 5-ASA, also called as mesalamine or mesalazine) or its derivatives have been used successfully for a relatively long time for the treatment of intestinal disorders, such as, for example, ulcerative colitis and Crohn's disease.

Ulcerative colitis (UC) is an idiopathic, chronic relapsing and remitting, non-specific inflammatory disease of the colonic mucosa. Acute episodes are characterized by chronic diarrhea, rectal bleeding and abdominal pain. Stool volume correlates directly with disease severity, since the colon becomes increasingly unable to reabsorb water and electrolytes as inflammation of the mucosa increases. Loss of water and electrolytes can lead to dehydration, weight loss and serum electrolyte disturbances. Inflammation of the mucosa leads to erosions, which eventually result in rectal bleeding. Anemia and hypoalbuminemia often develop as the disease progresses. Mucosal Inflammation also leads to smooth muscle spasm that, in turn, causes urgency to defecate and tenesmus. Systemic manifestations include anorexia, weight loss, fatigue, fever, increased sedimentation rate, arthritis, eye inflammation, anxiety, tachycardia, and elevation in liver function tests (LFTs).

UC also has a profound emotional and social impact on the affected individual. The etiology and pathogenesis of UC are multifactorial and incompletely understood. One theory is that the disease results from inappropriate activation of the mucosal immune system, resulting in the inflammatory response. Theories regarding the inappropriate activation suggest a role for genetic predisposition and/or environment triggers.

UC is most commonly reported in Northern Europe and the United States; reported less frequently in the Middle East and the Southern Hemisphere; and infrequently seen in South America, Asia and Africa. The annual incidence rate is 10.4 to 12.0 cases per 100,000 people with a prevalence rate of 35 to 100 cases per 100,000 people. Although UC occurs at any age, the incidence peaks at 15 to 25 years and 55 to 65 years. The disease is 30% more predominant in females; and a higher incidence is associated with the Jewish population. The goal of treatment in UC is to induce and maintain remission, and improve quality of life.

Subjects with ulcerative colitis may experience periods of remission (times when the symptoms go away) that can last for months or years. However, most subjects' symptoms eventually return. Active therapy is treatment given to treat UC symptoms when they are active. Maintenance therapy refers to treatment given to subjects to enable them to stay in remission, to maintain their health in a disease-free, or limited-disease state. Maintenance medications must be taken for a prolonged period of time.

The use of 5-ASA and its derivatives as a chemotherapeutic agent in colonic cancer is likewise known, polyps in the colon and rectum being associated with an increased risk of carcinoma (WO 95/18622).

A coloscopic polypectomy in patients with polyps in the colon and/or rectum results in a considerable reduction in risk of the formation of colonic carcinomas and is recommended as a therapy, in particular in the case of colorectal polyps. The recurrence rate after polypectomy, however, is high and amounts to about 6-30% per year. Aminosalicylic acid is suitable for the longer-term treatment of such patients and lowers the recurrence rate of colorectal polyps.

The action of aminosalicylic acid in the treatment of intestinal disorders, or in the prevention of their recurrence or in the prevention of secondary disorders arising therefrom and possible accompanying disorders takes place by means of the contact of the active compound directly at the site of the disorder in the intestine, the action of the aminosalicylic acid, or a derivative thereof, being directly related to its local concentration in the intestinal area to be treated.

Since inflammatory processes often affect relatively large sections of the intestinal tract, the pharmaceutical form should spread reproducibly over wide areas of the intestine and release the active compound only at the site of inflammation.

A problem in the treatment with aminosalicylic acid is that the active compound is very easily absorbed and can be excreted via the kidney before its action can occur.

FR 2 692 484 discloses a tablet for the controlled release of 4-ASA in a hydrophilic matrix which consists of swellable polymers forming a gel barrier, and having an enteric coating. After dissolution of the coating, the matrix swells and forms a gel barrier through which the active compound diffuses out. After an approximately two-hour lag phase, the composition disclosed in FR 2 692 484 releases the active compound approximately linearly in the intestine over a period of time of a further 14 hours.

U.S. Pat. Nos. 5,541,170 and 5,541,171 disclose an orally administrable solid pharmaceutical composition for the treatment of ulcerative colitis or Crohn's disease of the colon by selectively administering an effective amount of 5-aminosalicylic acid, or pharmaceutically acceptable salt or ester thereof, to the large intestine, said solid oral dosage form being coated with a layer which is insoluble in gastric juice and in intestinal juice below pH 7, but soluble in colonic intestinal juice, whereby the dosage form releases the 5-aminosalicylic acid, salt or ester to the right side of the colon.

U.S. Pat. No. 6,551,620 discloses an orally administrable pharmaceutical pellet formulation for the treatment of the intestinal tract, which comprises a core and an enteric coating, the core including, as a pharmaceutical active compound, aminosalicylic acid or a pharmaceutically tolerable salt or a derivative thereof and a non-gel matrix forming polymer.

U.S. Pat. No. 6,893,662 discloses a pharmaceutical composition in a solid unit dosage form for oral administration comprising mesalamine, an inner coating layer with a specific polymer and an outer coating layer with a specific polymer other than that used in the inner coating layer.

US Publication No. 2010/086588 discloses compositions and related methods for treating gastrointestinal disorders, e.g., inflammatory gastrointestinal disorders, irritable bowel disease, gastrointestinal motility disorders, functional gastrointestinal disorders, gastroesophageal reflux disease (GERD), Crohn's disease, ulcerative colitis, diverticulitis, inflammatory bowel disease, and gastroparesis, with a granulated mesalamine formulation. It discloses granulated mesalamine formulation in the form of a capsule. Also included in the publication are methods to extend remission of ulcerative colitis by administration of a once-daily dosage of granulated mesalamine.

U.S. Pat. No. 6,004,581 discloses an oral modified release composition comprising individually coated granules of mesalamine, each granule comprising: a core comprising 5-aminosalicylic acid (5-ASA) (or a salt or an ester thereof) and a spheronization aid, in particular microcrystalline cellulose, and a coating comprising a semi-permeable polymer, in particular, ethylcellulose. It discloses the granules ready to use being essentially spherical as defined by an aspect ratio within 1.00-1.25.

PCT Publication No. WO 2010/077908 discloses modified release formulations containing 5-aminosalicylic acid. It discloses a plurality of pellets or mini-tablets having 5-aminosalicylic acid and an enteric coating. The publication also discloses that the pellets or mini-tablets may be filled into a capsule. It also discloses formulation with beads having a size ranging from 800-1000 microns.

US Publication No. 2010/210605 discloses kits with compositions comprising 5-amino-2-hydroxybenzoic acid, a reducing sugar such as lactose and a predetermined amount of desiccant. The publication discloses that desiccants used in the compositions containing reducing sugar are useful in kits for preventing formation of the degradant 5-[2-formyl-5-hydroxymethyl-1H-pyrrol-1-yl]-2-hydroxybenzoic acid in compositions comprising 5-amino-2-hydroxybenzoic acid.

Pharmaceutical manufacturers in the art have heretofore used mesalamine raw materials to form mesalamine medications without regard to the density of the raw material fractions used. The result of this practice is that the manufacturers have been unable to produce a mesalamine capsule for strengths greater than 500 mg per dosage form.

Mesalamine has been marketed in the USA in different kinds of dosage forms for a number of years. The delayed release tablets of mesalamine are being marketed as Lialda® by Shire (1.2 gm mesalamine), Asacol® by Warner-Chilcott (400 mg mesalamine) and Asacol HD® by Warner-Chilcott (800 mg). The extended release capsules of mesalamine are available as Apriso® by Salix (375 mg mesalamine), Pentasa® by Shire (250 mg and 500 mg mesalamine) and Delzicol® by Warner-Chilcott (400 mg mesalamine). The dosage forms for rectal administration are also available such as Canasa®, a rectal suppository by Aptalis Pharma and Rowasa® enema by Meda Pharms. The capsule dosage form is preferred over the tablet dosage form due to several advantages of the capsule dosage form such as unique mixes and ingredients are possible; sealed hard capsule shells can be good oxygen barriers; protection for sensitive ingredients; the shell normally breaks down/opens in 4 minutes; reduced gastrointestinal irritation and odorless, tasteless, easy to swallow, etc.

There is still a need to develop an alternate stable capsule composition for human consumption wherein hard gelatin capsule shells can be used without compromising stability of the final composition. The current invention relates to a stable capsule dosage form of mesalamine wherein the hard gelatin capsule shell is filled with at least one tablet having mesalamine, wherein the tablet is enteric coated and the composition is devoid of any reducing sugar. The invention also relates to a preparation for obtaining such a composition.

### SUMMARY OF THE INVENTION

In one general aspect there is provided a stable pharmaceutical composition comprising: a hard gelatin capsule shell and at least one tablet within the capsule shell, wherein the tablet comprises mesalamine and one or more pharmaceutically acceptable excipients, and the composition is devoid of any reducing sugar or sugar alcohol.

In one embodiment, the composition is devoid of a reducing sugar selected from monosaccharides such as lactose, maltose, galactose, glucose, fructose, ribose, xylose, etc.

In one embodiment, the composition is devoid of a sugar alcohol selected from glycerol, erythritol, threitol, xylitol, ribitol, mannitol, sorbitol, galactitol, fucitol, iditol, isomalt, maltitol, lactitol, etc.

In another general aspect there is provided a stable capsule of mesalamine wherein the capsule is substantially free of 5-[2-formyl-5-hydroxymethyl-1H-pyrrol-1-yl]-2-hydroxybenzoic acid upon storage of the capsules at a relative humidity of 60% and a temperature of 25° C. for a period of at least six months.

In another aspect there is provided a stable capsule comprising mesalamine, wherein the capsule shell has more than about 10% by weight moisture content at a relative humidity of 60% and a temperature of 25° C.

In another aspect there is provided a capsule containing at least one tablet of mesalamine wherein the tablet comprises an intimate admixture of mesalamine and one or more pharmaceutically acceptable excipients.

In another aspect there is provided a stable pharmaceutical composition comprising mesalamine, wherein the mesalamine has a bulk density between about 0.3 g/ml and 0.8 g/ml.

Embodiments of the pharmaceutical composition may include one or more of the following features. For example, the pharmaceutically acceptable excipient may include a diluent(s), a disintegrant(s), a binder(s), a lubricant(s), a glidant(s), an antiadherent(s), a plasticizer(s), a solvent(s) and the like.

In another general aspect there is provided a kit comprising a pharmaceutical composition of mesalamine as per the invention; and a predetermined amount of desiccant.

In still another general aspect there is provided a method of improving the handling properties of a composition of mesalamine comprising the steps of: providing at least one tablet containing mesalamine having an enteric coat; and encapsulating the at least one tablet in a hard gelatin capsule shell.

In still another general aspect there is provided a method of improving the stability of a composition of mesalamine comprising the steps of: providing at least one tablet containing mesalamine having an enteric coat; and encapsulating at least one tablet in a hard gelatin capsule shell, wherein the capsule shell has more than about 10% by weight moisture content at a relative humidity of 60% and a temperature of 25° C.

In still another general aspect there is provided a process for preparing a pharmaceutical composition wherein the process comprises the steps of: preparing a tablet comprising mesalamine and one or more pharmaceutically acceptable excipients; coating the tablet with an enteric polymer; and filling the coated tablet in a hard gelatin capsule; wherein the composition is devoid of any reducing sugar or sugar alcohol.

Embodiments of the pharmaceutical composition may include one or more of the following features. For example, the pharmaceutically acceptable excipient may include a diluent(s), a disintegrant(s), a binder(s), a lubricant(s), a glidant(s), an antiadherent(s), a plasticizer(s), a solvent(s) and the like.

In another general aspect there is provided a method of treatment of mildly to moderately active ulcerative colitis and for the maintenance of remission of ulcerative colitis which comprises administering to a human patient in need thereof the pharmaceutical composition of mesalamine as herein described.

In one embodiment the composition comprises an outer capsule defining an inner volume; and an inner caplet comprising 600-1200 mg mesalamine; the mesalamine having a bulk density between about 0.3 g/ml and 0.8 g/ml and being compressed to a caplet form dimensioned for fitting within and optimizing the inner volume of said capsule.

In another embodiment the capsule comprises 600-1200 mg mesalamine, wherein the mesalamine is capable to be compressed into a caplet form dimensioned for fitting within and optimizing the inner volume of said capsule.

In another embodiment the capsule of mesalamine comprises a caplet comprising at least 70% by weight of mesalamine, wherein the mesalamine is capable to be compressed to a caplet form dimensioned for fitting within and optimizing the inner volume of said capsule.

In another embodiment the capsule of mesalamine comprises a caplet dimensioned for fitting within and optimizing the inner volume of said capsule, wherein the total weight of the caplet is at least 900 mg.

In another embodiment aspect of the invention the capsule of mesalamine comprises a caplet comprising 600-1200 mg mesalamine, wherein the mesalamine is capable of being compressed into a caplet form dimensioned for fitting within and optimizing the inner volume of said capsule and the mesalamine has a bulk density between about 0.3 g/ml and 0.8 g/ml.

In another embodiment the caplet comprises an intimate admixture of mesalamine and one or more pharmaceutically acceptable excipients.

In another embodiment the capsule is a “00” size capsule and the caplet is dimensioned for fitting within and optimizing the inner volume of the “00” size capsule.

In another embodiment there is provided a premix comprising mesalamine and one or more pharmaceutically acceptable excipients, wherein the premix has a bulk density between about 0.3 g/ml and 0.8 g/ml, preferably between about 0.4 g/ml and 0.7 g/ml.

In still another embodiment, there is provided a process for preparing a pharmaceutical composition wherein the process comprising the steps of: preparing a premix comprising mesalamine and one or more pharmaceutically acceptable excipients so that the premix has a bulk density between 0.3 g/ml and 0.8 g/ml; processing the premix; compressing the processed premix into a caplet; coating the caplet with an enteric polymer; and filling the coated caplet in a capsule.

The details of one or more embodiments of the invention are set forth in the description below. Other features, objects and advantages of the invention will be apparent from the description.

### DETAILED DESCRIPTION OF THE INVENTION

The inventors have surprisingly found that stable compositions of mesalamine can be prepared by using even hard gelatin capsule shells. The inventors have also found that high dose of mesalamine can be filled into a hard gelatin capsule without compromising stability of the composition. As per the invention, stable pharmaceutical compositions of mesalamine are devoid of any reducing sugar especially lactose.

The inventors have surprisingly found that the pharmaceutical compositions of mesalamine covered by the present invention are substantially free of 5-[2-formyl-5-hydroxymethyl-1H-pyrrol-1-yl]-2-hydroxybenzoic acid, when the composition is stored at a relative humidity of 60% and a temperature of 25° C. for a period of at least six months.

The term “reducing sugar” used throughout the specification refers to monosaccharides such as lactose, maltose, galactose, glucose, fructose, ribose, xylose, etc.

The term “devoid of a reducing sugar” and the like used throughout the specification refers to a composition substantially free of a reducing sugar. Therefore, in one embodiment it is possible that small amounts of a reducing sugar may be present. In other embodiments, no reducing sugar is added to the composition when formulating the composition. In other embodiments, no reducing sugar is present in the composition.

The term “sugar alcohol” used throughout the specification refers to alcohols selected from glycerol, erythritol, threitol, xylitol, ribitol, mannitol, sorbitol, galactitol, fucitol, iditol, isomalt, maltitol, lactitol, etc.

The term “devoid of a sugar alcohol” and the like used throughout the specification refers to a composition substantially free of a sugar alcohol. Therefore, in one embodiment it is possible that small amounts of a sugar alcohol may be present. In other embodiments, no sugar alcohol is added to the composition when formulating the composition. In other embodiments, no sugar alcohol is present in the composition.

The term “mesalamine” used throughout the specification refers to not only mesalamine per se, but also its pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs and pharmaceutically acceptable prodrugs thereof. The amount of mesalamine used in the present invention is in the range from about 200 to about 1200 mg in a single or divided dose. In a preferred embodiment of the present invention, the capsule dosage form comprises one tablet having 400 mg, 600 mg or 800 mg mesalamine.

Important physicochemical characteristics of powders are the density properties such as bulk and tapped density, weight variation and flow properties such as angle of repose. Bulk density is the undisturbed packing density of that substance and tapped density relates to the packing density after tapping a bed of substance until no change in the packing density is seen. Bulk density and tapped density, can be determined using compendial bulk density apparatus, such as the method given in Test 616 “Bulk Density and Tapped Density,” United States Pharmacopeia 29, United States Pharmacopeial Convention, Inc., Rockville, Md., 2005 (“USP”).

Bulk density of mesalamine used to prepare the tablet/caplet is preferably monitored before production to ensure that the bulk density of the mesalamine is at least about 0.3 g/ml and preferably from about 0.3 g/ml to about 0.8 g/ml, more preferably from about 0.4 g/ml to about 0.7 g/ml. This physical property of the drug is very important for making the tablet/caplet that can be filled into suitable sized capsules for human consumption. In another embodiment, if mesalamine having a bulk density lower than about 0.3 g/ml is used to prepare the composition, the mesalamine is treated with pharmaceutically acceptable excipients to make a premix having higher bulk density and thus ultimately the tablet/caplet can be filled into a proper sized capsule.

The composition of the present invention may include a high dose of mesalamine having a specific bulk density. The mesalamine composition is dimensioned to form a caplet for fitting within a capsule in a manner that optimizes the volume of the capsule, i.e., fills the internal volume of the capsule substantially completely.

Insertion of the mesalamine composition within the capsule masks the unpleasant and unpalatable taste of the enclosed caplet.

Hard-shell capsules offer a customized dosage form that can be made easily and conveniently in the pharmacy. Size #00 (double zero) is usually the largest capsule size used orally for humans. Hard-shell capsules are generally filled with powder or multiparticulates like granules, pellets, minitablets, microtablets, beads etc. Hard-shell capsules used in this invention are made up of gelatin. The capsule shell may be purchased from commercially available sources or made according to known methods. Hard capsule shells used in the present invention are made up of gelatin. Mesalamine of a proposed bulk density can be compressed to meet the target weight and fit into a “00” size capsule, without an unacceptable incidence of “capping” (i.e., splitting along a plane parallel to the long axis of the capsule) and “picking” (i.e., loss of small punctuate flecks of material from its surface). The capsule size used for the purpose of this invention may be selected from “Oel”, “Oel+”, “Oxel”, “00” or “00el”. The caplet should be dimensioned such that it should fit within the capsule to optimize its inner volume. The caplets may be prepared such that the length should not exceed 22 mm, width should not exceed 7.3 mm and thickness should not exceed 6.6 mm. In other embodiments, the ratio of length to width of the caplet should not be more than 3.1. The capsules may be filled with tablets having different shapes like oval shape, modified capsule shape, etc. Tablets with a modified caplet shape are preferred. These gelatin capsule shells have moisture content of more than 10% by weight at a relative humidity of 60% and a temperature of 25° C. Moisture content is expressed as the equilibrium moisture content and may be determined using any conventional technique.

A “delayed release” composition may be designed to delay the release of the drug for a specified period. Delayed release compositions of the present invention include those that exhibit a delayed-release, e.g., compositions that only begin releasing the drug after a fixed period of time. The delayed release compositions of the present invention may include the compositions which may release substantially no drug within two hours and after completion of the first two hours, the composition may release more than 80% of the drug within next two hours. The composition may release less than about 50%, preferably less than 30%, more preferably less than 10% of total drug within one hour after administration.

Hard gelatin capsules as per the invention may be filled with at least one tablet comprising mesalamine and one or more pharmaceutically acceptable excipients. The tablet may be in the form of a conventional tablet, a caplet, a mini-tablet, a microtablet, a bilayer tablet or a trilayer tablet. The capsule may be filled with one, two, three, four or five conventional tablets or with more than 10 mini-tablets. The conventional tablet may have a diameter of at least 3 mm while mini-tablets may have diameter up to 2 mm. The total number of tablets filled in the capsule depends on the dose of mesalamine per unit dosage form. Alternatively, hard gelatin capsules may be filled with pellets or granules of mesalamine. These pellets or granules may be prepared as per the techniques generally known in the art.

As used herein, the term “excipient” means any component admixed with or co-incorporated with the active agent. Excipients may act to facilitate incorporation of the active agent into the substrate, modify the release of the active agent from the substrate, stabilize the active agent, enhance absorption of the active agent, enhance tableting, increase the bulk of the pharmaceutical composition, and the like. The excipients are safe for their intended use at the levels employed in the formulation and are compatible with the active agent.

The pharmaceutically acceptable excipients may include one or more of a diluent(s), a disintegrant(s), a binder(s), a lubricant(s), a glidant(s), an antiadherent(s), a plasticizer(s) or combinations thereof. Excipients according to the composition of the invention do not include reducing sugars and sugar alcohols.

Suitable diluent(s) include, but are not limited to, microcrystalline cellulose, starch, dibasic calcium phosphate, tribasic calcium phosphate, calcium carbonate, dextrose, kaolin, magnesium carbonate, magnesium oxide; and mixtures thereof. The diluent may be added to increase the bulk volume of the powder to facilitate granulation or compression.

Suitable disintegrant(s) include, but are not limited to, croscarmellose sodium, crospovidone, sodium starch glycolate, corn starch, potato starch, maize starch and modified starches, calcium silicates, low substituted hydroxypropylcellulose or combinations thereof. The amount of disintegrating agent is preferably in the range of 5% to 35% by weight of the composition.

Suitable binder(s) include, but are not limited to, hydroxyethyl cellulose, hydroxypropyl cellulose, hydroxypropyl methylcellulose, carbomers, dextrin, ethyl cellulose, methylcellulose, shellac, zein, gelatin, polymethacrylates, polyvinyl pyrrolidone, corn starch, potato starch, maize starch, pregelatinized starch, sodium alginate, gums, synthetic resins and the like.

Suitable lubricant(s), glidant(s) or anti-adherent agent(s), include, but are not limited to, talc, metallic stearates such as magnesium stearate, calcium stearate, zinc stearate; colloidal silicon dioxide, finely divided silicon dioxide, stearic acid, hydrogenated vegetable oil, glyceryl palmitostearate, glyceryl monostearate, glyceryl behenate, polyethylene glycols, powdered cellulose, starch, sodium stearyl fumarate, sodium benzoate, mineral oil, magnesium trisilicate, kaolin; and mixtures thereof. It should be appreciated that a person skilled in the art is cognizant of the fact that lubricant(s), glidant(s) and anti-tacking agent(s) generally may be used interchangeably. The lubricant(s), glidant(s) or anti-tacking agent(s) may be present in an amount ranging from 0.1% to 10% by weight of the composition.

Suitable plasticizer(s), include, but are not limited to triacetin, diethyl phthalate, dibutyl sebecate, tributyl sebecate, acetyl tributyl citrate, polyethylene glycol or mixtures thereof. Preferably, the composition may be devoid of dibutyl phthalate.

The core tablet/caplet may be coated with the delayed release coating. The delayed release property of the dosage form may be achieved by using an enteric polymer. An “enteric polymer” used in the invention may be selected from the group consisting of hydroxypropyl methylcellulose phthalate, cellulose acetate phthalate, cellulose acetate succinate, methylcellulose phthalate, hydroxypropyl methylcellulose acetate succinate (HPMCAS), hydroxyethylcellulose phthalate, polyvinylacetate phthalate, polyvinyl butyrate acetate, vinyl acetate-maleic anhydride copolymer, styrene-maleic mono-ester copolymer, carboxymethyl ethylcellulose, poly(methacrylic acid, methyl methacrylate) 1:2 (Eudragit S-100), poly(methacrylic acid, methyl methacrylate) 1:1 (Eudragit L-100), methacrylic acid-ethyl acrylate copolymer (Eudragit L100-55 (dried methacrylic acid copolymer LD) or Eudragit L30D-55 (methacrylic acid copolymer LD)), methacrylic acid-methyl acrylate-methyl methacrylate copolymer (Eudragit FS30D), shellac or combinations thereof. Preferred enteric polymers used in the coating is poly(methacrylic acid, methyl methacrylate) 1:2 (Eudragit S-100), poly(methacrylic acid, methyl methacrylate) 1:1 (Eudragit L-100) or combinations thereof.

The core tablet/caplet may be coated with a barrier or protective layer. In a further embodiment the barrier layer comprises a non-toxic edible polymer, edible pigment particles, an edible polymer plasticizer, and a surfactant. Materials include, for example and without limitation, materials described in U.S. Pat. No. 4,543,370, incorporated herein by reference. Exemplary barrier and/or protective layers include OPADRY®; OPADRY II® which comprises HPMC, titanium dioxide, plasticizer and other components (e.g. OPADRY® Blue, OPADRY® Clear 03F59016, OPADRY® Green 03B510016); and polyvinyl alcohol-polyethylene glycol copolymer marketed as Kollicoat® IR. Suitable barrier layers, for illustration and without limitation, include Kollicoat IR (a polyvinyl alcohol-polyethylene glycol graft copolymer) and Kollicoat IR White®.

The thickness of the barrier and/or protective layer can vary over a wide range, but is generally in the range 20 to 3,000 microns, such as on the order of about 25 to 250 microns. Preferably the barrier layer retards the release of mesalamine by less than 5 minutes, preferably less than 4 minutes and more preferably by less than 3 minutes. The barrier and/or protective layer in the pharmaceutical composition may be prepared by using a suitable material such that it substantially inhibits the release of the active from their respective compartments in the gastric environment of pH below 4.5.

As per one of the embodiments, a stable pharmaceutical composition of mesalamine comprises at least one enteric coated tablet filled into a hard gelatin capsule shell, wherein the composition is devoid of any reducing sugar or sugar alcohol. In another embodiment, a capsule comprises mesalamine in an amount from 200 mg to 1200 mg and one or more pharmaceutically acceptable excipients. The tablet/caplet may comprise an intimate mixture of mesalamine and one or more pharmaceutically acceptable excipients.

Another embodiment of the invention relates to the stable capsule dosage form of mesalamine wherein the dosage form is devoid of 5-[2-formyl-5-hydroxymethyl-1H-pyrrol-1-yl]-2-hydroxybenzoic acid upon storage of the capsules at a relative humidity of 60% and a temperature of 25° C. for a period of at least six months. In this embodiment, the dosage form comprises hard gelatin capsule shells with a moisture content of more than 10% by weight at a relative humidity of 60% and at a temperature of 25° C.

In still another embodiment, a hard gelatin capsule may comprise mesalamine in an amount from 400 mg to 800 mg and one or more pharmaceutically acceptable excipients. In this embodiment, a premix of mesalamine is compressed to a caplet form dimensioned for fitting within and optimizing the inner volume of the capsule. The caplet may comprise an intimate mixture of mesalamine and one or more pharmaceutically acceptable excipients. The premix of mesalamine has a bulk density preferably from about 0.3 g/ml to about 0.8 g/ml, more preferably from about 0.4 g/ml to about 0.7 g/ml. As an alternate, mesalamine having a bulk density between about 0.3 g/ml and 0.8 g/ml may be used as an active ingredient in preparing a core tablet.

The particles of the premix may have a mean particle size of less than 250 microns, preferably less than 200 microns, more preferably less than 150 microns and most preferably less than 100 microns.

The pharmaceutical compositions as described herein may be prepared by processes known to the person having ordinary skill in the art of pharmaceutical technology such as direct compression, wet granulation, dry granulation or melt granulation.

In another embodiment, a caplet may be obtained by compressing coated multiparticulates. For this embodiment, multiparticulates may be in the form of pellets, granules or beads prepared by the techniques known in the art. Pellets may be prepared by an extrusion-spheronization process. Granules may be prepared by wet granulation, dry granulation or melt granulation process. Beads may be prepared by coating inert carriers like sugar spheres or MCC spheres with a solution or dispersion containing the drug. These multiparticulates may be coated with a seal coat/barrier coat/intermediate coat/protective coat and then may be coated with an enteric coat to provide delayed release multiparticulates. The coating layer may comprise some excipients which may provide cushioning effect to the particles which may give protection against breakage of coating during compression.

In another embodiment, a pharmaceutical composition of mesalamine may be prepared by the process wherein the process comprising the steps of: preparing a mixture comprising mesalamine and one or more pharmaceutically acceptable excipients; processing the mixture; compressing the processed mixture into a tablet; coating the tablet with an enteric polymer; and filling the coated tablet in a capsule.

In another embodiment, a capsule comprising mesalamine may be prepared by providing mesalamine having a bulk density between 0.3 g/ml and 0.8 g/ml; mixing the mesalamine with one or more pharmaceutically acceptable excipients; compressing the mixture into a tablet/caplet; coating the tablet/caplet with an enteric polymer; and filling the coated caplet in a capsule.

In another embodiment, a caplet of mesalamine may be prepared by mixing and granulating mesalamine having a bulk density between 0.3 g/ml and 0.8 g/ml with one or more pharmaceutically acceptable excipients; drying the granules; lubricating the dried granules; and compressing the granules into a caplet. In another embodiment, a caplet may be prepared by mixing mesalamine having a bulk density between 0.3 g/ml and 0.8 g/ml with one or more pharmaceutically acceptable excipients; lubricating the powder mixture; and compressing the powder into a caplet. In still another embodiment, a caplet may be prepared by mixing mesalamine having a bulk density between 0.3 g/ml and 0.8 g/ml with one or more pharmaceutically acceptable excipients; making slugs of the mixture by dry compression technique; breaking the slugs to provide the granules; lubricating the granules; and compressing the granules into a caplet.

In another embodiment, a caplet may be obtained by compressing coated multiparticulates. For this embodiment, multiparticulates may be in the form of pellets, granules or beads prepared by the techniques known in the art. Pellets may be prepared by extrusion-spheronization process. Granules may be prepared by wet granulation, dry granulation or melt granulation process. Beads may be prepared by coating inert carriers such as sugar spheres or MCC (microcrystalline cellulose) spheres with a solution or dispersion containing the mesalamine. These multiparticulates may be coated with a seal coat/barrier coat/intermediate coat/protective coat and then may be coated with an enteric coat to provide delayed release multiparticulates. The coating layer may comprise some excipients which may provide cushioning effect to the particles which may give protection against breakage of coating during compression.

The dosage forms of the embodiments of the present invention are preferably packaged as part of a kit which comprises at least one of the present dosage forms and a predetermined amount of a desiccant. Therefore, another embodiment of the invention is directed to such a kit.

As used herein, a “desiccant” is a material that will absorb moisture by physical and/or chemical means. Activated desiccants are desiccants that have been treated by heating and ventilating, or by other means, to develop an internal surface on which moisture and certain vapors or gases may be collected. Examples of the “desiccant” to be used in the present invention include activated carbon, calcium chloride, metallic oxide, such as an alkaline earth metallic oxide (e.g. calcium oxide (CaO) etc.), an alkaline earth metallic hydroxide (e.g. calcium hydroxide etc.), sulfate of an alkaline earth metal (e.g. magnesium sulfate, calcium sulfate etc.), silicon dioxide (silica gel), a bonded product of alumina oxide and silicon dioxide (silica alumina), alumina oxide (active alumina), natural or synthetic zeolite (molecular sieves 3A, 4A, SA, 13X), allophane, clay, a mixture of clay and activated carbon, a mixture of silica gel and activated carbon, a mixture of silica gel and clay, a mixture of silica alumina and activated carbon, a mixture of synthetic zeolite and activated carbon, a mixture of allophane and activated carbon (e.g., allophane added with activated carbon, or allophane kneaded with activated carbon etc.), pulp containing silica gel (e.g., ultrafine silica gel mixed between paper fibers, silica gel packaged in paper tube etc.), pulp containing calcium chloride (e.g., paper material impregnated with liquid calcium chloride, dried and coated with film etc.), pulp containing allophane (e.g., pulp impregnated with allophane liquid, dried and film coated, allophane packaged in paper tube etc.) and the like.

Desiccant quantities may be adjusted depending on the need of the application. For example, a bulk product may be stored in a drum with a suitable amount of a desiccant, or it may first be divided into smaller batches and stored appropriately with a smaller quantity of a desiccant. The moisture initially in a package from the drug substance, excipients, and fillers, as well as moisture permeation into the package over its shelf life, will determine the amount of desiccant required in a given package. The quantity of desiccant in an ideal case should be at least sufficient to absorb this moisture and maintain a desiccated environment within the package for the product's shelf life.

The amount of a desiccant required may depend upon the number of unit dosage forms to be packaged per container and the water absorptive capacity of the desiccant. Typically, the predetermined desiccant quantity is in excess of the desiccant required.

In still other embodiment, there is provided a method of treatment of mildly to moderately active ulcerative colitis and for the maintenance of remission of ulcerative colitis which comprises administering to a human patient in need thereof the pharmaceutical composition of mesalamine as per the invention.

In another embodiment, the composition of the present invention comprising mesalamine exhibits bioequivalence to a reference composition of mesalamine. As used herein, a “reference composition” is intended to mean a composition of mesalamine which is currently approved as Asacol HD in the United States and which may be used as a reference for a new drug application (NDA) or an abbreviated new drug application (ANDA) under the Federal Food Drug & Cosmetic Act.

The bioequivalence studies were carried out between Asacol HD® tablets (reference) and compositions of the invention (test) in fasted and fed state. The study was monitored in terms of Cₘₐₓ and AUC achieved with the test product and the reference product (Asacol HD®).

In further embodiment, the present invention provides a stable pharmaceutical composition comprising mesalamine, wherein the composition comprises a hard gelatin capsule shell and at least one tablet within the capsule shell; wherein the composition is devoid of any reducing sugar or sugar alcohol and when administered as two dosage units three times daily for six days provides an in-vivo plasma profile for mesalamine with a mean of Cₘₐₓ ranging from 1 μg/mL to 9 μg/mL, a mean of AUC₀₋ₜ ranging from 6 μg*hr/mL to 34 μg*hr/mL; and a mean of Tₘₐₓ ranging from 10 to 16 hours.

The absorbed mesalamine is rapidly acetylated in the gut mucosal wall and by the liver to N-acetyl-5-aminosalicylic acid (N-Ac-5-ASA) which is excreted mainly by the kidney. In still further embodiments, the present invention provides a stable pharmaceutical composition comprising mesalamine, wherein the composition comprises a hard gelatin capsule shell and at least one tablet within the capsule shell; wherein the composition is devoid of any reducing sugar or sugar alcohol; and when administered as two dosage units three times daily for six days provides an in-vivo plasma profile for active metabolite N-acetyl-5-aminosalicylic acid with a mean of Cₘₐₓ ranging from 2 μg/mL to 7 μg/mL, a mean of AUC₀₋ₜ ranging from 14 μg*hr/mL to 36 μg*hr/mL; and a mean of Tₘₐₓ ranging from 10 to 16 hours.

In a further embodiment, the present invention provides a pharmaceutical composition comprising mesalamine, wherein the composition comprises an outer capsule defining an inner volume; an inner caplet comprising 600-1200 mg mesalamine; and when administered as two dosage units three times daily for six days provides an in-vivo plasma profile for mesalamine with a mean of Cₘₐₓ ranging from 1 μg/mL to 9 μg/mL, a mean of AUC₀₋ₜ ranging from 6 μg*hr/mL to 34 μg*hr/mL; and a mean of Tₘₐₓ ranging from 10 to 16 hours.

The invention is further illustrated by the following examples which are provided to be exemplary of the invention and not to limit the scope of the invention.

### Example 1

| Sr. No | Ingredient | % w/w |
|---|---|---|
| Granulation | | |
| 1 | Mesalamine | 75.47 |
| 2 | Sodium starch glycolate | 2.26 |
| 3 | Micro-crystalline cellulose | 9.79 |
| 4 | Polyvinylpyrrolidone | 1.89 |
| 5 | Purified water | q.s. |
| Extragranular | | |
| 6 | Sodium starch glycolate | 3.40 |
| 7 | Talc | 0.49 |
| 8 | Colloidal silicon dioxide | 0.52 |
| 9 | Magnesium stearate | 0.52 |
| Coating Formulation | | |
| 10 | Methacrylic acid copolymer (Eudragit S100) | 3.68 |
| 11 | Talc | 0.63 |
| 12 | Acetyl tributyl citrate | 1.10 |
| 13 | Titanium dioxide | 0.24 |
| 14 | Ferric oxide | 0.01 |
| 15 | Isopropyl alcohol | q.s. |
| 16 | Purified water | q.s. |

### Process:

Mesalamine, sodium starch glycolate and microcrystalline cellulose were mixed together and granulated with an aqueous solution of povidone. The granules were dried and dried granules were milled. The milled granules were mixed with remaining quantity of sodium starch glycolate, talc and colloidal silicon dioxide. The mixture was lubricated with magnesium stearate. The lubricated mixture was then compressed to provide a tablet. This tablet was filled into a hard gelatin capsule shell.

### Example 2

| Sr. No | Ingredient | % w/w |
|---|---|---|
| Compaction | | |
| 1 | Mesalamine | 70-80 |
| 2 | Colloidal silicon dioxide | 0.2-2.0 |
| 3 | Magnesium stearate | 0.2-2.0 |
| 4 | Sodium starch glycolate | 1-5 |
| Granulation | | |
| 5 | Microcrystalline Cellulose | 8-15 |
| 6 | Polyvinylpyrrolidone | 1-5 |
| 7 | Purified water | q.s. |
| Lubrication | | |
| 8 | Sodium starch glycolate | 1-5 |
| 9 | Colloidal silicon dioxide | 0.5-2.0 |
| 10 | Talc | 0.5-2.0 |
| 11 | Magnesium stearate | 0.3-2.0 |
| Coating Formulation | | |
| 12 | Methacrylic acid copolymer | 1.0-8.0 |
| | (Eudragit S) | |
| 13 | Acetyl tributyl citrate | 0.1-2.0 |
| 14 | Talc | 0.1-1.0 |
| 15 | Titanium dioxide | 0.1-1.0 |
| 16 | Isopropyl alcohol | q.s. |

### Process:

### Preparation of a Premix:

Mesalamine, sodium starch glycolate, colloidal silicon dioxide and magnesium stearate were mixed and compacted to provide a premix.

### Preparation of a Caplet:

The premix thus prepared was mixed with microcrystalline cellulose and was granulated with aqueous solution of polyvinylpyrrolidone to provide granules of mesalamine. The granules were dried and lubricated. The granules were compressed with proper tooling to provide caplets. The caplets thus formed were coated with an enteric coating dispersion of methacrylic acid copolymer.

### Preparation of a Capsule:

The enteric coated caplet was filled in the capsule shell to provide a final capsule dosage form.

### Example 3

| | | Quantity |
|---|---|---|
| Sr. No | Ingredient | (mg/tablet) |
| Granulation | | |
| 1 | Mesalamine | 800 |
| 2 | Microcrystalline cellulose | 100 |
| 3 | Polyvinylpyrrolidone | 21 |
| 4 | Sodium starch glycolate | 36 |
| 5 | Purified water | q.s. |
| Extragranular | | |
| 6 | Sodium starch glycolate | 26 |
| 7 | Colloidal silicon dioxide | 6 |
| 8 | Talc | 5.8 |
| 9 | Magnesium stearate | 5.2 |
| Coating Formulation | | |
| 10 | Methacrylic acid copolymer | 40.80 |
| | (Eudragit S 100 | |
| 11 | Acetyl tributyl citrate | 11.54 |
| 12 | Talc | 7.12 |
| 13 | Titanium dioxide | 1.8 |
| 14 | Isopropyl alcohol | q.s. |
| 15 | Ferric oxide (Red) | 1.10 |

### Process:

### Preparation of Granules:

Mesalamine, sodium starch glycolate and microcrystalline cellulose were mixed together and the powder mixture was granulated using aqueous solution of polyvinylpyrrolidone.

### Preparation of a Caplet:

The granules were dried and mixed with sodium starch glycolate, talc and colloidal silicon dioxide. The blend was lubricated with magnesium stearate. The lubricated granules were compressed with proper tooling to provide caplets. The caplets thus formed were coated with an enteric coating dispersion of methacrylic acid copolymer.

### Preparation of a Capsule:

The enteric coated caplet was filled in the capsule shell to provide a final capsule dosage form.

Mesalamine used to prepare the composition as per Example 3 has a bulk density of 0.483 g/ml and a tapped density 0.694 g/ml. The capsule used to fill the caplet was size “00el”.

While the present invention has been described in terms of its specific embodiments, certain modifications and equivalents will be apparent to those skilled in the art and are intended to be included within the scope of the present invention.

1. A stable pharmaceutical composition comprising:
(a) a hard gelatin capsule shell; and
(b) at least one tablet within the capsule shell, wherein the tablet comprises mesalamine and one or more pharmaceutically acceptable excipients,
wherein the composition is devoid of any reducing sugar or sugar alcohol.

2. The stable pharmaceutical composition according to claim 1, wherein the composition is substantially free of 5-[2-formyl-5-hydroxymethyl-1H-pyrrol-1-yl]-2-hydroxybenzoic acid upon storage of the composition at a relative humidity of 60% and a temperature of 25° C. for a period of at least six months.

3. The stable pharmaceutical composition according to claim 1, wherein the tablet contains an enteric coat that prevents release of the mesalamine in the upper gastrointestinal tract.

4. The stable pharmaceutical composition according to claim 3, wherein the enteric coat comprises poly(methacrylic acid, methyl methacrylate) at a ratio of about 1:2, poly(methacrylic acid, methyl methacrylate) at a ratio of about 1:1, or a mixture thereof.

5. The stable pharmaceutical composition according to claim 1, wherein the capsule shell has more than about 10% by weight moisture content at a relative humidity of 60% and a temperature of 25° C.

6. The stable pharmaceutical composition according to claim 1, wherein the tablet comprises from about 200 mg to about 1200 mg of mesalamine.

7. The stable pharmaceutical composition according to claim 1, wherein the mesalamine has a bulk density of between about 0.3 g/ml and 0.8 g/ml.

8. The stable pharmaceutical composition according to claim 1, wherein the tablet is greater than 5 mm in diameter.

9. The stable pharmaceutical composition according to claim 1, wherein the capsule shell contains one tablet.

10. The stable pharmaceutical composition according to claim 1, wherein the reducing sugar comprises one or more of lactose, maltose, galactose, glucose, fructose, ribose and xylose.

11. The stable pharmaceutical composition according to claim 1, wherein the sugar alcohol comprises one or more of glycerol, erythritol, threitol, xylitol, ribitol, mannitol, sorbitol, galactitol, fucitol, iditol, isomalt, maltitol, and lactitol.

12. A stable pharmaceutical composition of mesalamine comprising a hard gelatin capsule shell and at least one tablet within the capsule shell; wherein the composition is devoid of any reducing sugar or sugar alcohol; and when administered as two capsules three times daily for six days provides an in-vivo plasma profile for mesalamine with a mean of Cₘₐₓ ranging from 1 μg/mL to 9 μg/mL, a mean of AUC₀₋ₜ ranging from 6 μg*hr/mL to 34 μg*hr/mL; and a mean of Tₘₐₓ ranging from 10 to 16 hours.

13. A stable pharmaceutical composition of mesalamine comprising a hard gelatin capsule shell and at least one tablet within the capsule shell; wherein the composition is devoid of any reducing sugar or sugar alcohol; and when administered as two capsules three times daily for six days provides an in-vivo plasma profile for the active metabolite N-acetyl-5-aminosalicylic acid with a mean of Cₘₐₓ ranging from 2 μg/mL to 7 μg/mL, a mean of AUC₀₋ₜ ranging from 14 μg*hr/mL to 36 μg*hr/mL; and a mean of Tₘₐₓ ranging from 10 to 16 hours.

14. A kit comprising:
the stable pharmaceutical composition according to claim 1; and
a predetermined amount of desiccant.

15. The kit according to claim 14, wherein the desiccant is silica gel.
